(19) United States
(12) Patent Application Publication
(10) Pub. No.: US 2022/0145282 A1
(43) Pub. Date: May 12, 2022

(54) Cell-Free Compositions for ATP Regeneration and Uses Thereof

(71) Applicant: BIOCHEMINSIGHTS, INC., Malvern, PA (US)

(72) Inventors: William Armiger, Malvern, PA (US); David Dodds, Malvern, PA (US)

(21) Appl. No.: 17/430,634

(22) PCT Filed: Feb. 12, 2020
(86) PCT No.: PCT/US20/17991

§ 371 Date: Aug. 12, 2021

(51) Int. Cl. C12N 11/04; C12N 9/14

(52) U.S. Cl. CPC C12N 11/04; C12Y 306/03014; C12N 9/14

(57) ABSTRACT

A method of using an electrochemical cell, specifically a membrane bioreactor, to provide electrons to an electron transport chain capable of generating a proton gradient for performing ATP regeneration from ADP. Such an electron transport chain may be part of, or contained within, a synthetic membrane, or may be prepared by the suitable disruption of living cells. Electrons provided by the electrochemical cell are passed to the electron transport system via a suitable electron carrier, such as NADH2, FMNH2, FADH2, reduced ubiquinone(s), thiols, or other electron carriers or biological reducing equivalents that are compatible with the components of the electron transport chain performing ATP regeneration.

### CROSS REFERENCE TO RELATED APPLICATIONS

This application claims priority to and the benefit of U.S. Provisional Application No. 62/804,448 filed Feb. 12, 2019, the disclosure of which is incorporated herein by reference in its entirety.

### TECHNICAL FIELD

The present disclosure relates generally to cell-free systems for the regeneration of adenosine triphosphate (ATP), widely required in various biologically mediated reactions such as the synthesis of peptides and proteins.

### BACKGROUND

Cell-free synthesis is now recognized as a powerful method for producing valuable commercial compounds via biological processes, without the need for practising classical fermentation, and various methods for regenerating ATP for the purpose of allowing cell-free systems to perform ATP-requiring actions, such as the coupling of amino acids in peptide synthesis, have also been described. (“Development of Prokaryotic Cell-Free Systems for Synthetic Biology”; Abel C. Chiao, Richard M. Murray, Zachary Z. Sun, doi: http://dx.doi.org/10.1101/048710).

The major practical issue, after constructing the desired metabolic pathways in the cell-free system, is supplying the chemical potential energy necessary to drive the desired metabolic pathways. This chemical potential energy is generally provided as the common naturally occurring adenosine triphosphate (ATP). Providing a source of ATP to the cell-free system is a considerable practical problem which severely limits the utility of cell-free systems.

Most simply, ATP can be provided directly to the cell-free system as a purified compound, in stoichiometric amounts. This is extremely expensive, even at bench-scale, and not economically feasible at commercial scale.

Providing ATP to cell-free systems can be achieved by including additional metabolic pathways together with the desired metabolic pathways of the cell-free system. These additional pathways regenerate ATP from ADP within the cell free system using less expensive reagents, such as glucose, pyruvate, succinate, glutamate, or other chemical compounds which are added to the overall cell-free system in stoichiometric amounts. These reagents serve the role of “sacrificial substrates” and are acted upon by the additional pathways added to the pathways initially present in the cell-free system. But these additional pathways are generally not part of the pathway of the cell-free system which directly provides the desired product of the cell-free system. Thus, these additional pathways introduce an additional complexity to the overall cell-free system, with attendant inefficiencies and expense.

Thus, a need exists for improved cell-free systems and methods for the regeneration of ATP in vitro.

### SUMMARY

In one aspect, provided herein is a cell-free composition for regenerating adenosine triphosphate (ATP) comprising:

a reduced chemical species for delivering electrons to an electron acceptor, wherein said reduced chemical species is electrochemically generated by an electrochemical device;

an electron transport chain (ETC) comprising the electron acceptor and ETC complex IV, wherein the electron acceptor is selected from the group consisting of ETC complex I, ETC complex II, ETC complex III, ubiquinone and cytochrome c, wherein said ETC is capable of generating a proton gradient across a lipid bilayer;

an ATP synthase capable of utilizing the proton gradient across the lipid bilayer to produce ATP from adenosine diphosphate (ADP); and

a liposome enclosed by the lipid bilayer to retain protons within the liposome, wherein the ETC and the ATP synthase are associated with the lipid bilayer.

A further aspect relates to a device for regenerating adenosine triphosphate (ATP), comprising:

an anode contained in an anode chamber and a cathode contained in a cathode chamber;

deionized water in the anode chamber in contact with the anode;

a proton permeable membrane that separates the anode and cathode chambers;

a liquid phase in the cathode chamber continuously in contact with the cathode, said liquid phase comprising an electron transfer mediator (ETM) capable of undergoing cyclic reduction and oxidation, wherein, once reduced at the cathode, the ETM is reduced into reduced chemical species that can transfer electrons to an electron acceptor of an electron transport chain (ETC); wherein the electron acceptor is selected from the group consisting of ETC complex I, ETC complex II, ETC complex III, ubiquinone and cytochrome c; wherein the ETC comprising the electron acceptor and ETC complex IV;

a synthetic mitochondrion comprising an enclosed lipid bilayer, the ETC, and an ATP synthase; wherein said ETC is capable of generating a proton gradient across the lipid bilayer; wherein said ATP synthase capable of utilizing the proton gradient across the lipid bilayer to produce ATP from adenosine diphosphate (ADP); wherein the enclosed lipid bilayer is capable of retaining protons therein;

a process stream containing a substrate capable of using the generated ATP for producing a desired product; and

an external power source providing a voltage between the anode and the cathode.

In some embodiments, the reduced chemical species can include one or more of NADH2, NADPH2, FMNH2, FADH2 and/or ubiquinol. In certain embodiments, the reduced chemical species is one or more of NADH2, NADPH2 and/or FMNH2, and the electron acceptor is ETC complex I. In certain embodiments, the reduced chemical species is FADH2, and the electron acceptor is ETC complex II. In certain embodiments, the reduced chemical species is ubiquinol, and the electron acceptor is ETC complex III.

In certain embodiments, the ETC has been reconstituted into the liposome to at least partially embed in the lipid bilayer, with the electron acceptor accessible to the reduced chemical species that are present outside the liposome. In certain embodiments, the ATP synthase has been reconstituted into the liposome to at least partially embed in the lipid bilayer, and wherein the ATP synthase is accessible to the ADP that is present outside the liposome. In some embodiments, the ETC and the ATP synthase are reconstituted to be associated with, preferably at least partially embedded within, the lipid bilayer.

In certain embodiments, the lipid bilayer is impermeable to protons. In certain embodiments, the liposome is artificially prepared. In certain embodiments, the ETC and/or ATP synthase are recombinantly produced.

In some embodiments, the device can include a membrane located between the cathode and the process stream, wherein said membrane supports the synthetic mitochondrion and permits interfacial contact between the liquid phase and the process stream, while preventing substantial mixing of the liquid phase and the process stream and preventing the process stream from substantially contacting the cathode. In some embodiments, the membrane is configured to partially associate with the synthetic mitochondrion. In some embodiments, the membrane is configured such that the synthetic mitochondrion is partially or completely embedded within the membrane.

Also provided herein are methods of using the compositions and devices disclosed herein, to regenerate ATP in vitro.

### BRIEF DESCRIPTION OF THE DRAWINGS

FIG. 1 illustrates the comparative simplicity of providing electrons to the ETC by electrochemical regeneration of NADH2 compared to providing reducing equivalents from additional chemical compounds and metabolic pathways.

FIG. 2 illustrates, in a schematic, the components of the electron transport chain (ETC) and ATP synthase, contained by a biological membrane, or the membrane of a prepared liposome.

FIG. 3 illustrates, in a schematic, the electrochemical reduction of NAD to NADH2 and the provision of NADH2 to a synthetic mitochondrion which utilizes the NADH2 to transform ADP to ATP via the ETC and ATP synthase.

FIG. 4 illustrates, in a schematic, the electrochemical reduction of NAD to NADH2 and the provision of NADH2 to a synthetic mitochondrion which is contained within the matrix of a membrane.

### DETAILED DESCRIPTION

In some embodiments, the present disclosure makes use of electrochemical devices for reducing NAD to NADH2 via the electrolysis of water (e.g., the Electrochemical Bioreactor Module (EBM) as disclosed in PCT publication numbers WO2017160793A1, WO2016137976A1, WO2016070168A1 and WO2014039767A1, all incorporated herein by reference). NADH2 is subsequently consumed by a synthetic mitochondrion and returned to NAD, from which NADH2 is electrochemically regenerated, and aberrant forms of NADH2 are recovered as NAD for subsequent reduction. In this manner, no stoichiometric amounts of sacrificial substrates are needed to provide NADH2, or to provide reducing equivalents generally to a biological system.

### Definitions

Unless defined otherwise, all technical and scientific terms used herein have the same meaning as commonly understood by those of ordinary skill in the art to which this invention pertains.

As used herein, the articles “a” and “an” refer to one or more than one, e.g., to at least one, of the grammatical object of the article. The use of the words “a” or “an” when used in conjunction with the term “comprising” herein may mean “one,” but it is also consistent with the meaning of “one or more,” “at least one,” and “one or more than one.”

As used herein, “about” and “approximately” generally mean an acceptable degree of error for the quantity measured given the nature or precision of the measurements. Exemplary degrees of error are within 20 percent (%), typically, within 10%, and more typically, within 5% of a given range of values. The term “substantially” means more than 50%, preferably more than 80%, and most preferably more than 90% or 95%.

As used herein the term “comprising” or “comprises” is used in reference to compositions, methods, and respective component(s) thereof, that are present in a given embodiment, yet open to the inclusion of unspecified elements.

As used herein the term “consisting essentially of” refers to those elements required for a given embodiment. The term permits the presence of additional elements that do not materially affect the basic and novel or functional characteristic(s) of that embodiment of the disclosure.

The term “consisting of” refers to compositions, methods, and respective components thereof as described herein, which are exclusive of any element not recited in that description of the embodiment.

As used herein, “a plurality of” means more than 1, e.g., 2, 3, 4, 5, 6, 7, 8, 9, 10, 11, 12, 13, 14, 15, 16, 17, 18, 19, 20, or more, e.g., 25, 30, 40, 50, 60, 70, 80, 90, 100, 200, 300, 400, 500, or more, or any integer therebetween.

A “cell-free” system is a system that is able to conduct certain biological reactions outside of the context of a cell.

A “liposome” or “synthetic mitochondrion”, used interchangeably, refers to a vesicle or a microscopic particle formed by at least one lipid bilayer. The liposomes may be artificially prepared. In some embodiments, the liposomes can have an average diameter of about 50-900 nm, about 50-500 nm, about 60-480 nm, about 80-450 nm, about 100-400 nm, about 50-300 nm, about 80-250 nm, or about 100-200 nm.

As used herein, the term “lipid” refers to any of a group of organic compounds, including the fats, oils, waxes, sterols, and triglycerides, that are insoluble in water but soluble in nonpolar organic solvents, are oily to the touch, and together with carbohydrates and proteins constitute the principal structural material of living cells.

Examples of suitable lipids for forming the liposome include, but are not limited to: phosphatidylcholines such as 1,2-dioleoyl-phosphatidylcholine, 1,2-dipalmitoyl-phosphatidylcholine, 1,2-dimyristoylphosphatidylcholine, 1,2-distearoyl-phosphatidylcholine, 1-oleoyl-2-palmitoylphosphatidylcholine, 1-oleoyl-2-stearoyl-phosphatidylcholine, 1-palmitoyl-2-oleoyl, phosphatidylcholine and 1-stearoyl-2-oleoyl-phosphatidylcholine; phosphatidylethanolamines such as 1,2-dioleoyl-phosphatidylethanolamine, 1,2-dipalmitoyl-phosphatidylethanolamine, 1,2-dimyristoyl-phosphatidylethanolamine, 1,2-distearoyl-phosphatidylethanolamine, 1-oleoyl-2-palmitoyl-phosphatidylethanolamine, 1-oleoyl-2-stearoyl-phosphatidylethanolamine, 1-palmitoyl-2-oleoyl phosphatidylethanolamine, 1-stearoyl-2-oleoyl-phosphatidylethanolamine and succinyl-dioleoyl-phosphatidylethanolamine; phosphatidylserines such as 1,2-dioleoylphosphatidylserine, 1,2-dipalmitoyl-phosphatidylserine, 1,2-dimyristoylphosphatidylserine, 1,2-distearoyl-phosphatidylserine, 1-oleoyl-2-palmitoylphosphatidylserine, 1-oleoyl-2-stearoyl-phosphatidylserine, 1-palmitoyl-2-oleoylphosphatidylserine and 1-stearoyl-2-oleoyl-phosphatidylserine; phosphatidylglycerol such as 1,2-dioleoyl-phosphatidylglycerol, 1,2-dipalmitoyl-phosphatidylglycerol, 1,2-dimyristoyl-phosphatidylglycerol, 1,2-distearoyl-phosphatidylglycerol, 1-oleoyl-2-palmitoyl-phosphatidylglycerol, 1-oleoyl-2-stearoyl-phosphatidylglycerol, 1-palmitoyl-2-oleoyl-phosphatidylglycerol and 1-stearoyl-2-oleoyl-phosphatidylglycerol; pegylated lipids; pegylated phospoholipids such as phophatidylethanolamine-N-[methoxy(polyethyleneglycol)-1000], phophatidylethanolamine-N-[methoxy(polyethyleneglycol)-2000], phophatidylethanolamine-N-[methoxy(polyethyleneglycol)-3000], phophatidylethanolamine-N-[methoxy(polyethyleneglycol)-5000]; pegylated ceramides such as N-octanoylsphingosine-1-{succinyl[methoxy(polyethyleneglycol)1000]}, N-octanoyl-sphingosine-1-{succinyl[methoxy(polyethyleneglycol)2000]}, N-octanoyl-sphingosine-1-{succinyl[methoxy(polyethyleneglycol)3000]}, N-octanoyl-sphingosine-1-{succinyl[methoxy(polyethyleneglycol)5000]}; lyso-phosphatidylcholines, lysophosphatidylethanolamines, lyso-phosphatidylglycerols, lyso-phosphatidylserines, ceramides; sphingolipids; glycolipids such as ganglioside GM1; glucolipids; sulphatides; phosphatidic acid, such as di-palmitoyl-glycerophosphatidic acid; palmitic fatty acids; stearic fatty acids; arachidonic fatty acids; lauric fatty acids; myristic fatty acids; lauroleic fatty acids; physeteric fatty acids; myristoleic fatty acids; palmitoleic fatty acids; petroselinic fatty acids; oleic fatty acids; isolauric fatty acids; isomyristic fatty acids; isostearic fatty acids; sterol and sterol derivatives such as cholesterol, cholesterol hemisuccinate, cholesterol sulphate, and cholesteryl-(4-trimethylammonio)-butanoate, ergosterol, lanosterol; polyoxyethylene fatty acids esters and polyoxyethylene fatty acids alcohols; polyoxyethylene fatty acids alcohol ethers; polyoxyethylated sorbitan fatty acid esters, glycerol polyethylene glycol oxy-stearate; glycerol polyethylene glycol ricinoleate; ethoxylated soybean sterols; ethoxylated castor oil; polyoxyethylene polyoxypropylene fatty acid polymers; polyoxyethylene fatty acid stearates; dioleoyl-sn-glycerol; dipalmitoyl-succinylglycerol; 1,3-dipalmitoyl-2-succinylglycerol; 1-alkyl-2-acyl-phosphatidylcholines such as 1-hexadecyl-2-palmitoylphosphatidylcholine; 1-alkyl-2-acyl-phosphatidylethanolamines such as 1-hexadecyl-2-palmitoyl-phosphatidylethanolamine; 1-alkyl-2-acyl-phosphatidylserines such as 1-hexadecyl-2-palmitoyl-phosphatidylserine; 1-alkyl-2-acyl-phosphatidylglycerols such as 1-hexadecyl-2-palmitoyl-phosphatidylglycerol; 1-alkyl-2-alkyl-phosphatidylcholines such as 1-hexadecyl-2-hexadecyl-phosphatidylcholine; 1-alkyl-2-alkylphosphatidylethanolamines such as 1-hexadecyl-2-hexadecylphosphatidylethanolamine; 1-alkyl-2-alkyl-phosphatidylserines such as 1-hexadecyl-2-hexadecyl-phosphatidylserine; 1-alkyl-2-alkyl-phosphatidylglycerols such as 1-hexadecyl-2-hexadecyl-phosphatidylglycerol; N-Succinyl-dioctadecylamine; palmitoylhomocysteine; lauryltrimethylammonium bromide; cetyltrimethyl-ammonium bromide; myristyltrimethylammonium bromide; and poly(dimethylsiloxane)-graft-poly(ethylene oxide) (PDMS-g-PEO).

An “electron transport chain” (ETC) is a series of complexes that transfer electrons from electron donors to electron acceptors via redox (both reduction and oxidation occurring simultaneously) reactions, and couples this electron transfer with the transfer of protons (H⁺ ions) across a membrane. This creates an electrochemical proton gradient that drives the synthesis of adenosine triphosphate (ATP) by ATP synthase.

The term “proton gradient” across the liposome membrane or lipid bilayer, as used herein, refers to a difference in proton concentration between the solution on one side of the liposome membrane and the solution on the other side of the liposome membrane.

As used herein, the term “electron transfer mediator” or “ETM” means a molecule capable of accepting one or more electrons itself, and then transferring electrons to another molecule, including the transfer of electrons to an enzyme molecule. A typical and well known ETM is Neutral Red, which is also used as a pH indicator. Other compounds that can function as an ETM include Methylene Blue, Methyl Viologen, and quinone. Most generally, and compound whose reduction potential is more negative than that of NAD can be used, and this includes a variety of compounds generally termed redox dyes. For example, in the situation previously described, the Neutral Red is acting as an electron transport mediator by facilitating the movement of electrons from the cathode to the NAD cofactor, thus facilitating the reduction of the NAD to produce NADH2.

The term “electron transport mediator” or “ETM” can include molecules that facilitate the transfer of electrons to an enzyme molecule, thus in a broad sense cofactors (e.g., NADH2, FMN, FAD, ferredoxin, etc.) may also be considered an electron transfer mediator. However, in some examples, the term “electron transport mediator” or ETM is meant to describe only those molecules which facilitate the transfer of electrons, but which are not otherwise generally considered to be the naturally occurring cofactors of redox enzyme systems, for example, NADH, FMN, FAD, ferredoxin and the like.

In the present disclosure, the descriptor “NAD(P)” indicates the oxidized state of the phosphorylated and non-phosphorylated forms of the nicotinamide adenine dinucleotide, while the descriptor “NAD(P)H₂” indicates the reduced state of the phosphorylated and non-phosphorylated forms of the nicotinamide adenine dinucleotide.

### ETC and ATP Production

In order to generate ATP, electrons are passed through a series of cellular components collectively called the electron transport chain (ETC), itself embedded in a membrane. This passage of electrons through the ETC creates a proton concentration gradient across the membrane. The proton gradient is then used to drive the reaction by which ATP is regenerated from adenosine diphosphate (ADP). The ATP is then available for any metabolic process that may be part of the desired cell-free system, for example, a cell-free system by which proteins are synthesized, and specifically in this example, the reaction which activates the amino acid bound to the tRNA specific to that amino acid, allowing it to undergo amide bond formation and thus extend the amino acid chain during the synthesis of a protein. Other biological reactions requiring ATP, such as the phosphorylation of carbohydrate molecules, also utilize ATP generated by the creation of a proton gradient by the ETC.

Electrons are provided to the ETC by molecules generally known as biological reducing equivalents. These are most generally nicotinamide adenine dinucleotide (NAD), flavin adenine dinucleotide (FAD) and ubiquinone. In order for the ETC to generate the proton gradient necessary to perform ATP formation from ADP, these molecules must be provided in their chemically reduced forms; i.e., NADH2, FADH2 and ubiquinol, respectively. Each of these chemically reduced molecules, that is, biological reducing equivalents, is accepted by different parts of the ETC, but in all cases, the ETC will generate a proton gradient, and allow the ATP synthase to produce ATP from ADP.

The ETC itself is a collection of proteins that are contained or embedded within a membrane, and it is across this membrane that a proton gradient will be generated by the action of the proteins that form the ETC. These proteins are most commonly:

NADH-ubiquinone oxidoreductase, also called “complex I”

Succinate-ubiquinone oxidoreductase, also called “complex II”

Ubiquinone-cytochrome c oxidoreductase, also called “complex III”

Cytochrome c, also called “cyt c”

Cytochrome c oxidase, also called “complex IV”

In complex I (NADH:ubiquinone oxidoreductase, NADH-CoQ reductase, or NADH dehydrogenase; EC 1.6.5.3), two electrons are removed from NADH and ultimately transferred to a lipid-soluble carrier, ubiquinone (UQ). The reduced product, ubiquinol (UQH₂), freely diffuses within the membrane, and Complex I translocates four protons (H⁺) across the membrane, thus producing a proton gradient. Complex I is one of the main sites at which premature electron leakage to oxygen occurs, thus being one of the main sites of production of superoxide. The pathway of electrons is as follows: NADH is oxidized to NAD⁺, by reducing Flavin mononucleotide to FMNH₂ in one two-electron step. FMNH₂ is then oxidized in two one-electron steps, through a semiquinone intermediate. Each electron thus transfers from the FMNH₂ to an Fe—S cluster, from the Fe—S cluster to ubiquinone (Q). Transfer of the first electron results in the free-radical (semiquinone) form of Q, and transfer of the second electron reduces the semiquinone form to the ubiquinol form, QH₂. During this process, four protons are translocated from the mitochondrial matrix to the intermembrane space. As the electrons become continuously oxidized and reduced throughout the complex an electron current is produced along the 180 Angstrom width of the complex within the membrane. This current powers the active transport of four protons to the intermembrane space per two electrons from NADH.

In complex II (succinate dehydrogenase or succinate-CoQ reductase; EC 1.3.5.1) additional electrons are delivered into the quinone pool (Q) originating from succinate and transferred (via flavin adenine dinucleotide (FAD)) to Q. Complex II consists of four protein subunits: succinate dehydrogenase, (SDHA); succinate dehydrogenase [ubiquinone] iron-sulfur subunit, mitochondrial, (SDHB); succinate dehydrogenase complex subunit C, (SDHC) and succinate dehydrogenase complex, subunit D, (SDHD). Other electron donors (e.g., fatty acids and glycerol 3-phosphate) also direct electrons into Q (via FAD). Complex II is a parallel electron transport pathway to complex I, but unlike complex I, no protons are transported to the intermembrane space in this pathway. Therefore, the pathway through complex II contributes less energy to the overall electron transport chain process.

In complex III (cytochrome bc₁ complex or CoQH₂-cytochrome c reductase; EC 1.10.2.2), the Q-cycle contributes to the proton gradient by an asymmetric absorption/release of protons. Two electrons are removed from QH₂ at the Qₒ site and sequentially transferred to two molecules of cytochrome c, a water-soluble electron carrier located within the intermembrane space. The two other electrons sequentially pass across the protein to the Qᵢ site where the quinone part of ubiquinone is reduced to quinol. A proton gradient is formed by one quinol oxidation at the Qₒ site to form one quinone at the Qᵢ site.

In complex IV (cytochrome c oxidase; EC 1.9.3.1), sometimes called cytochrome AA3, four electrons are removed from four molecules of cytochrome c and transferred to molecular oxygen (O₂), producing three molecules of water. At the same time, eight protons are removed from the mitochondrial matrix (although only four are translocated across the membrane), contributing to the proton gradient.

ATP synthase is the protein which uses the proton gradient created a cross the membrane by the ETC to drive the formation of ATP from ADP. The ETC can accept electrons from NADH2 at complex I, FADH2 at complex II, or ubiquinol (also known as Coenzyme Q10) at complex III. ATP synthase itself does not accept electrons from the ETC. It is embedded in the same membrane as the ETC, and utilizes the proton gradient that the ETC has generated across the membrane.

Ubiquinone may be reduced to ubiquinol by a stoichiometric amount of a suitable reagent, such as dithiothreitol (DTT), and the resulting ubiquinol used to provide electrons to the ETC. (“Toward Artificial Mitochondrion: Mimicking Oxidative Phosphorylation in Polymer and Hybrid Membranes”; Lado Otrin et al., Nano Lett. 2017, 17, 6816-6821; incorporated herein by reference.)

Electrons are passed from complex I or from complex II to complex III to then shuttled to Complex IV via cytochrome c (cyt c). Protons are driven across the membrane containing the ETC and ATP synthase at complex I, complex III, cyt c, and complex IV. At complex IV, the electrons leave the ETC and are used to reduce oxygen to water. (“A Cell-Free Framework for Rapid Biosynthetic Pathway Prototyping and Enzyme Discovery”, Ashty S. Karim, Michael C. Jewett; Metabolic Engineering 36 (2016) 116-126; incorporated herein by reference.)

Those skilled in the art will recognize that succinate can be provided to complex II, where it is oxidized to fumarate with concomitant reduction of FAD to FADH2. Those skilled in the art will also recognize that complex I includes the biological electron carrier flavin mononucleotide (FMN) which will also be present as its reduced from FMNH2, that complex III can include cytochrome b, and that cyt c is a relatively small, water soluble component that can be loosely associated with either ETC complex III or ETC complex IV.

### ATP Regeneration Using EBM and Synthetic Mitochondrion

Conventionally, ATP regeneration in vitro in cell-free systems can be achieved by including additional metabolic pathways. The added metabolic pathways include sufficient enzymes to generate reducing equivalents (e.g. NADH2 or NADPH2) from the added chemical reagents, which are in turn provided to the series of components that make up the ETC found in the membranes of mitochondria.

While including the ETC for performing ATP regeneration mitigates the problem of providing stoichiometric amounts of very expensive exogenous ATP to the desired cell-free system, it still requires stoichiometric reagents, that is, sacrificial substrates, and even more metabolic pathways. These further additional pathways are essential for transforming the less expensive stoichiometric reagents, such as glucose, pyruvate, succinate or glutamate, to the reducing equivalents that must be generated to carry electrons to the ETC. These additional pathways make the overall cell-free system more complex, leading to instability and other practical issues, such as the presence of both intermediates and end products of the enzymatic reactions generating the reducing equivalents, and the need to purify these away from the desired product of the cell-free system.

Thus, the present disclosure provides considerable practical advantage by providing the necessary biological reducing equivalents NADH2, FMNH2, FADH2 and ubiquinol directly to the ETC without the need to feed stoichiometric amounts of either expensive ATP, or of a sacrificial substrate (or mixtures of substrates) such as glucose, pyruvate, DTT, succinate or glutamate, and without the need to provide the additional metabolic pathways which generate the required NADH2, FMNH2, FADH2 and ubiquinol for the ETC from these sacrificial substrate materials. This would make the entire cell-free system simpler, more stable, and less expensive both to construct and to operate.

Thus, it is advantageous to provide the biological reducing equivalents required by the ETC, via electrochemical means as disclosed herein, to generate the proton gradient required for the transformation of ADP to ATP by ATP synthase, in a manner that does not require the use of stoichiometric sacrificial substrates or additional metabolic pathways or enzymes.

This can be accomplished by providing biological reducing equivalents via electrochemical methods such as those disclosed in PCT publication numbers WO2017160793A1, WO2016137976A1, WO2016070168A1 and WO2014039767A1, all incorporated herein by reference. Such methods remove the need to include metabolic pathways to generate biological reducing equivalents in cell-free systems.

More specifically, the present disclosure relates to use of the Electrochemical Bioreactor Module (EBM) for the purpose of regenerating ATP. ATP regeneration is achieved by providing reducing equivalents to a synthetic or artificial prepared membrane which contains ATP synthase and other components of the electron transport chain (ETC) that is normally found in the mitochondrion. The components of the ETC utilize the provided reducing equivalents to generate a proton gradient across the membrane, which allows the ATP synthase to regenerate ATP. The ATP thus generated can be used to generate other nucleotide triphosphates by exchange of the one or two of the pendant phosphate groups. This is a particular and novel advantage of the cell-free systems which seek to perform valuable metabolic processes without using the entire cellular metabolic machinery.

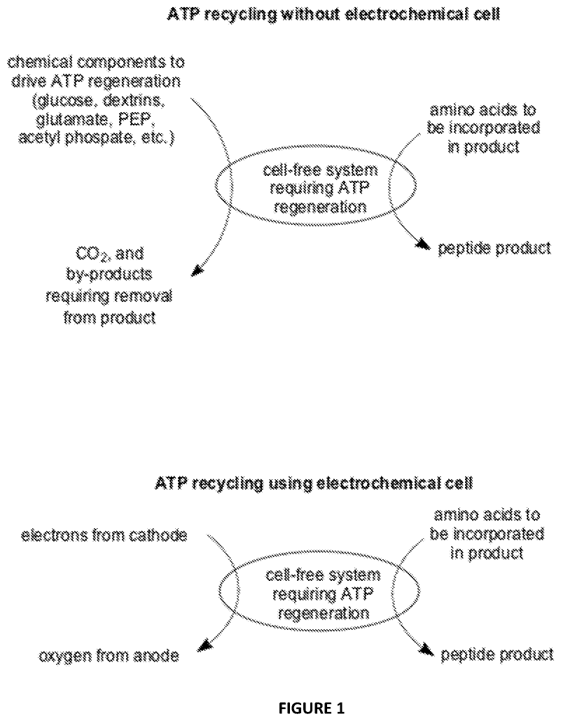

FIG. 1 illustrates the comparative simplicity of providing electrons to the ETC by electrochemical regeneration of NADH compared to providing reducing equivalents from additional chemical compounds and metabolic pathways.

In some embodiments, the present disclosure is directed to the use of an electrochemical device or system for providing electrons to an ETC, in association with ATP synthase in a manner capable of generating ATP using the electrons provided by electrochemically produced biological reducing equivalents. Specifically, the electrons are provided to the ETC by NADH2, NADPH2, FADH2 or reduced forms of ubiquinone which have been electrochemical generated. While these biological reducing equivalents are used as examples herein, as long as the reduced species provided to the synthetic mitochondrion have sufficient redox potential to transfer electrons at least to ETC complex IV, a proton gradient will be established, as shown by the use of DTT in Otrin et al.

In one embodiment, a cell-free system is arranged for the production of a desired material, for example, a protein, or other molecule requiring ATP to provide the chemical potential energy for its synthesis. This system requires ATP in order to operate, and the ATP is converted to ADP in the process.

ATP is produced in this embodiment by the ETC components complex I, complex II, complex III, cyt c and complex IV, plus ATP synthase. These are arranged in the lipid bilayer of a vesicle, also referred to as a “synthetic mitochondrion”, such that the ETC pumps protons from outside the vesicle to the interior of the vesicle as electrons move along the ETC, generating a proton gradient, which is used by the ATP synthase to generate ATP from ADP. The required electrons are provided to the ETC by NADH2 which has been electrochemically produced from NAD. Upon delivering the electrons to the ETC, the NADH2 is oxidized to NAD and the NAD is again converted to NADH2 by the EBM. The regenerated NADH2 again delivers electrons to the ETC, which again generates a proton gradient across the membrane of the vesicle, allowing the ATP synthase to regenerate ATP from the ADP resulting from the consumption of the ATP during the process of making the protein. In this embodiment, optionally FAD can be present and reduced to FADH2, delivering electrons to ETC complex II.

Thus, in this embodiment, no stoichiometric amount of biological reducing equivalent is required to provide electrons to the ETC, nor are any other metabolic steps for generating reducing equivalents from a sacrificial substrate required. As long as electrons are provided by the cathode of the electrochemical device, ATP will be generated, and the cell-free system will be provided with the chemical potential energy necessary to make the desired product.

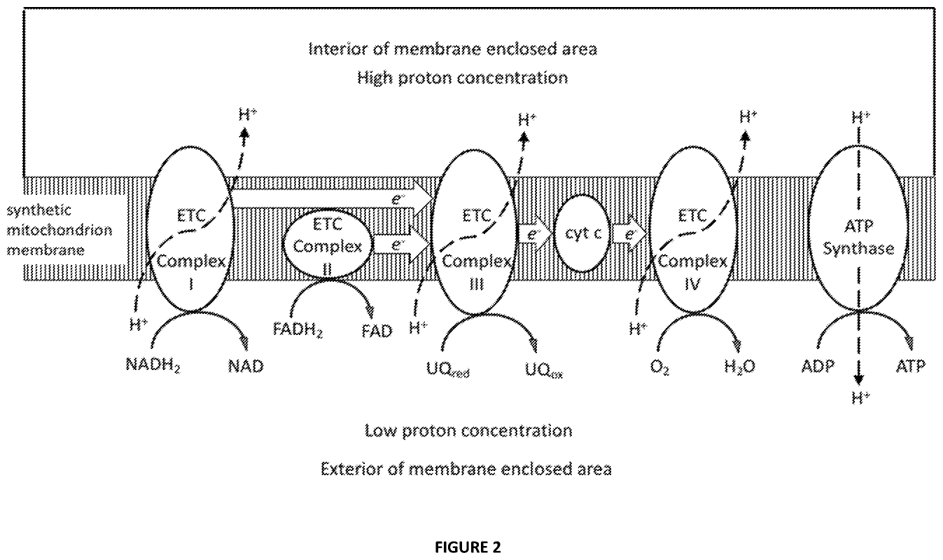

As illustrated in FIG. 2, the components of the ETC and ATP synthase can be contained by a biological membrane (e.g., a lipid membrane extracted from a biological source), or the lipid bilayer of an artificially prepared, non-naturally occurring liposome (used interchangeably with “synthetic mitochondrion”). The locations at which electrons may be delivered to the ETC, and the cofactors that can deliver them (NADH2, FADH2, reduced ubiquinone, UQred) are indicated. The general flow of electrons through the ETC, and of protons across the biological membrane, or the membrane of a prepared liposome, to generate a proton gradient, are also illustrated.

In certain embodiments, only ETC complex I, ETC complex III, cyt c and ETC complex IV are present, together with ATP synthase are present in the membrane of the synthetic mitochondrion. Electrons are delivered from NADH2 to ETC complex I and thence to ETC complex III.

In yet another embodiment, the synthetic mitochondrion, has only ATP synthase and an ETC composed only of complex III, cyt c and complex IV. The necessary biological reducing equivalents are provided by the electrochemical reduction of ubiquinone to ubiquinol, which delivers electrons to the abbreviated ETC. The resulting ubiquinone is passed through the EBM, and the ubiquinol regenerated.

By arranging for the ETC to be composed of different members of the groups of complex I, complex II, complex III, and complex IV (plus cytochromes b and c), the electrons from the EBM can be delivered by different biological reducing equivalents; e.g. NADH2, or FADH2 or ubiquinol.

As these different biological reducing equivalents can be generated at different cathode potentials in the EBM, there are operational advantages to the entire system, as a biological reducing equivalent can be chosen to deliver electrons to the ETC for the generation of ATP at a cathode potential that prevents unwanted, non-biological electrochemical reactions.

In some embodiments, the synthetic mitochondrion can include any liposomes or similar lipid bilayer containers or nanocontainers known in the art, such as those disclosed in Klara et al., J. Am. Chem. Soc. 2016, 138, 28-31; De Vocht et al., Controlled Release 2009, 137, 246-254; Stoenescu et al., Macromol. Biosci. 2004, 4, 930-935; Kumar et al., Proc. Natl. Acad. Sci. U.S.A. 2007, 104, 20719-20724; Jabůrek et al., Circ. Res. 2006, 99, 878-883; Nordlund et al., Nat. Commun. 2014, 5, 4303; von Ballmoos et al., Biochim. Biophys. Acta, Bioenerg. 2016, 1857, 321-323; Choi et al., Nano Lett. 2005, 5, 2538-2542; Otrin et al., Nano Lett. 2017, 17, 6816-6821; all incorporated herein by reference.

In some embodiments, the lipid bilayers can include various lipids such as phosphatidylcholines; phosphatidylethanolamines; phosphatidylserines; phosphatidylglycerol; pegylated lipids; pegylated phospoholipids; pegylated ceramides; lyso-phosphatidylcholines, lysophosphatidylethanolamines, lyso-phosphatidylglycerols, lyso-phosphatidylserines, ceramides; sphingolipids; glycolipids such as ganglioside GM1; glucolipids; sulphatides; phosphatidic acid; palmitic fatty acids; stearic fatty acids; arachidonic fatty acids; lauric fatty acids; myristic fatty acids; lauroleic fatty acids; physeteric fatty acids; myristoleic fatty acids; palmitoleic fatty acids; petroselinic fatty acids; oleic fatty acids; isolauric fatty acids; isomyristic fatty acids; isostearic fatty acids; sterol and sterol derivatives such as cholesterol, cholesterol hemisuccinate, cholesterol sulphate, and cholesteryl-(4-trimethylammonio)-butanoate, ergosterol, lanosterol; polyoxyethylene fatty acids esters and polyoxyethylene fatty acids alcohols; polyoxyethylene fatty acids alcohol ethers; polyoxyethylated sorbitan fatty acid esters, glycerol polyethylene glycol oxy-stearate; glycerol polyethylene glycol ricinoleate; ethoxylated soybean sterols; ethoxylated castor oil; polyoxyethylene polyoxypropylene fatty acid polymers; polyoxyethylene fatty acid stearates; dioleoyl-sn-glycerol; dipalmitoyl-succinylglycerol; 1,3-dipalmitoyl-2-succinylglycerol; 1-alkyl-2-acyl-phosphatidylcholines such as 1-hexadecyl-2-palmitoylphosphatidylcholine; 1-alkyl-2-acyl-phosphatidylethanolamines such as 1-hexadecyl-2-palmitoyl-phosphatidylethanolamine; 1-alkyl-2-acyl-phosphatidylserines such as 1-hexadecyl-2-palmitoyl-phosphatidylserine; 1-alkyl-2-acyl-phosphatidylglycerols such as 1-hexadecyl-2-palmitoyl-phosphatidylglycerol; 1-alkyl-2-alkyl-phosphatidylcholines such as 1-hexadecyl-2-hexadecyl-phosphatidylcholine; 1-alkyl-2-alkylphosphatidylethanolamines such as 1-hexadecyl-2-hexadecylphosphatidylethanolamine; 1-alkyl-2-alkyl-phosphatidylserines such as 1-hexadecyl-2-hexadecyl-phosphatidylserine; 1-alkyl-2-alkyl-phosphatidylglycerols such as 1-hexadecyl-2-hexadecyl-phosphatidylglycerol; N-Succinyl-dioctadecylamine; palmitoylhomocysteine; lauryltrimethylammonium bromide; cetyltrimethyl-ammonium bromide; myristyltrimethylammonium bromide; and poly(dimethylsiloxane)-graft-poly(ethylene oxide) (PDMS-g-PEO).

In some embodiments, the lipids are selected to prepare a lipid bilayer that is sufficiently tight and impermeable to protons, so as to retain the proton gradient generated by the ETC. The lipid bilayer, in some embodiments, has sufficient fluidity so as to facilitate the rotational motion of ATP synthase as well as the proton translocation by both the ETC (from outside to inside the liposome) and the ATP synthase (from inside to outside the liposome).

Liposomes can be prepared using methods known in the art, such as those disclosed in Akbarzadeh et al., Nanoscale Res Lett. 2013; 8(1): 102; Choi et al., Nano Lett. 2005, 5, 2538-2542; Otrin et al., Nano Lett. 2017, 17, 6816-6821; all incorporated herein by reference.

In various embodiments, the protein components of the synthetic mitochondrion (e.g., ETC complexes, ATP synthase) can be recombinantly produced. These proteins can be transmembrane proteins, which can be assembled into the liposomes such that they span the entire width of the lipid bilayer. In some embodiments, one or more of the proteins are at least partially embed in the lipid bilayer. In certain embodiments, assembly of the proteins can be achieved via reconstitution sequentially or simultaneously. For example, preformed liposomes can be mixed with ATP synthase and/or ETC in the presence of suitable detergents such as sodium cholate, sodium deoxycholate, octyl glucoside, or any combination of the foregoing. A broad concentration range of all detergents (e.g., 0.2-0.8% with 0.2% increments) can be screened for optimal reconstitution conditions. The reconstitution mixture can be incubated with mild agitation followed by various detergent removal methods such as using size exclusion column, by Bio-Beads or by dialysis.

In various embodiments, the proteins are functionally reconstituted. By “functional” it is meant that the protein, after reconstitution, should have the correct orientation across the lipid bilayer such that it is accessible to its substrates. For example, ETC and/or ATP synthase can be reconstituted into the liposome with proper orientation, so that the ETC can react with the reduced chemical species (e.g., NADH2) and oxygen that are present outside the liposome, and that the ATP synthase is accessible to the ADP that is present outside the liposome. Furthermore, the ETC needs to pump protons into the liposomes to build a proton gradient with higher proton concentration on the inside of the liposomes. The proton gradient needs to be sufficiently high to drive ATP synthase. Thus, in one embodiment, a protein-per-liposome ratio of about 1 ATP synthase and 1-10, 2-10, or 5 functional ETC components per liposome can be used. In some embodiments, to account for the random orientation of the reconstituted ETC, excess amount of the enzyme can be integrated into liposomes (e.g., 2-20 ETC components per liposome).

One liposome can have one or more ATP synthase and one or more ETC components. With increasing number of proteins per vesicle, higher ATP synthesis rates can be achieved.

### Devices for ATP Regeneration

Various devices for ATP regeneration are also provided herein. In some embodiments, a device can comprise:
an anode contained in an anode chamber and a cathode contained in a cathode chamber;
deionized water in the anode chamber in contact with the anode;
a proton permeable membrane that separates the anode and cathode chambers;
a liquid phase in the cathode chamber continuously in contact with the cathode, said liquid phase comprising an electron transfer mediator (ETM) capable of undergoing cyclic reduction and oxidation, wherein, once reduced at the cathode, the ETM is reduced into reduced chemical species that can transfer electrons to an electron acceptor of an electron transport chain (ETC); wherein the electron acceptor is selected from the group consisting of ETC complex I, ETC complex II, ETC complex III, ubiquinone and cytochrome c; wherein the ETC comprising the electron acceptor and ETC complex IV;
a synthetic mitochondrion comprising an enclosed lipid bilayer, the ETC, and an ATP synthase; wherein said ETC is capable of generating a proton gradient across the lipid bilayer; wherein said ATP synthase capable of utilizing the proton gradient across the lipid bilayer to produce ATP from adenosine diphosphate (ADP); wherein the enclosed lipid bilayer is capable of retaining protons therein;
a process stream containing a substrate capable of using the generated ATP for producing a desired product; and
an external power source providing a voltage between the anode and the cathode.

In some embodiments, the device can include the Electrochemical Bioreactor Module (EBM) as disclosed in PCT publication numbers WO2017160793A1, WO2016137976A1, WO2016070168A1 and WO2014039767A1, all incorporated herein by reference. In some embodiments, the proton permeable membrane of the device can be a modified Nafion (Trademark E.I DuPont) membrane which allows protons (as hydronium ions, H₃O+) to travel across it. The proton permeable membrane may support or contain a catalyst on the anode side, for the production of oxygen gas.

In some embodiments, the device can further include a membrane located between the cathode and the process stream, wherein said membrane supports the synthetic mitochondrion and permits interfacial contact between the liquid phase and the process stream, while preventing substantial mixing of the liquid phase and the process stream and preventing the process stream from substantially contacting the cathode. The membrane can be hydrophilic, and can have pores which extend from one surface of the membrane through to the other surface. The membrane can be any suitable thickness, e.g., from about 50 microns to 500 microns. The material composition of the membrane may be polysulfone (PS), polyethylene sulfone (PES), polyacrylamide (PA), polyacrylonitrile (PAN). The membrane may be a polysulfone (PS) with surface modification to impart hydrophilicity (Alenazi et al., Designed Monomers and Polymers, 2017 Vol. 20, no. 1, 532-546, incorporated by reference).

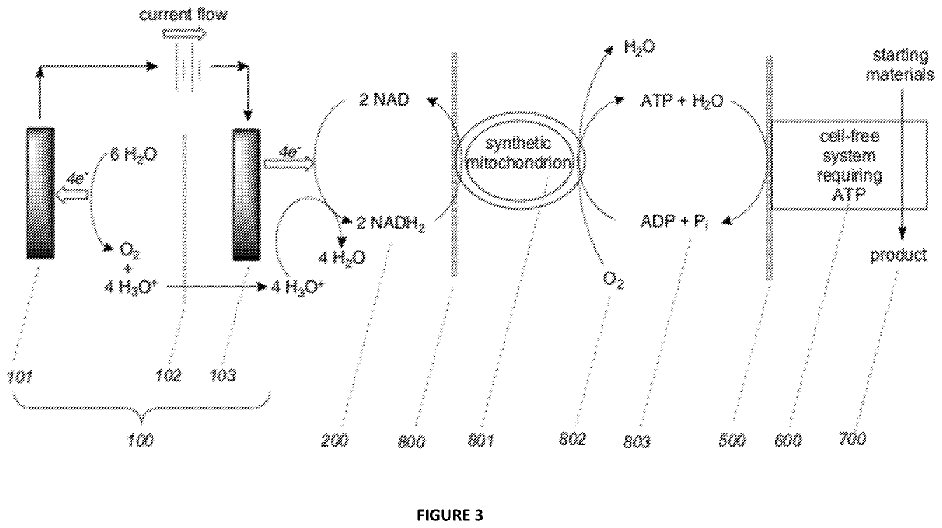

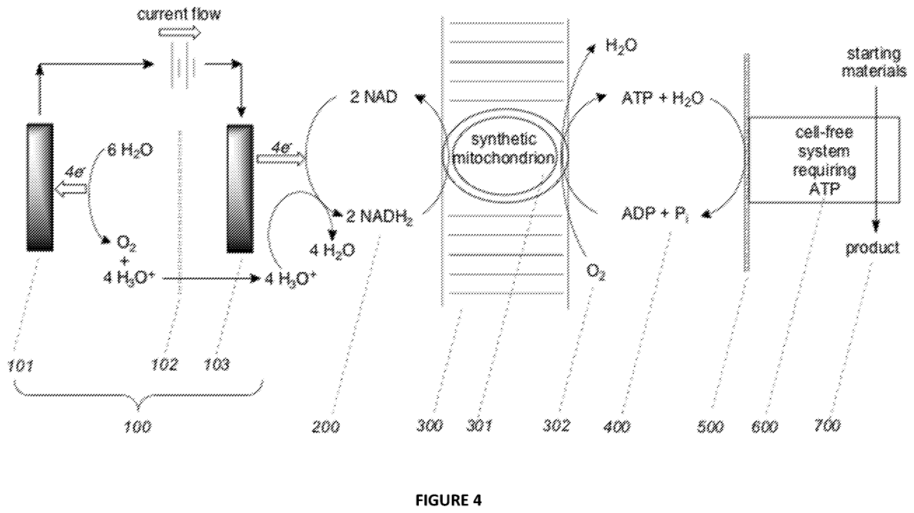

This membrane can be “asymmetric”, as the size of the pores can have openings that are larger on one side (e.g., the side facing the bulk reaction phase) than on the other side (e.g., the side facing the cathode) of the membrane. The size of the pores can be expressed as a molecular weight cutoff. For example, a membrane may be called a 10 KDa membrane, meaning that the size of the pore of the side of the membrane where the pore size is smaller is of a size such that molecule having a molecular weight greater than 10 KDa (kilodalton) will not pass through the membrane to any substantial degree. In some embodiments, the pores can have a first opening on a first surface of the membrane facing the cathode, wherein the first opening is sufficiently small to prevent molecules having a molecular weight greater than 100 KDa (e.g., greater than 100 kDa, greater than 80 kDa, greater than 60 kDa, greater than 50 kDa, greater than 40 kDa, greater than 30 kDa, greater than 20 kDa, greater than 20 kDa, greater than 10 kDa, greater than 5 kDa, or greater than 1 kDa) from substantially passing through. The pores can further have a second opening on a second surface of the membrane facing the process stream. In some embodiments, such as the membrane 801 in FIG. 3, the second opening is sufficiently small to only permit passing of NAD/NADH2 through the membrane. In some embodiments, such as the membrane 300 in FIG. 4, the second opening is sufficiently large to permit at least a portion of the synthetic mitochondrion contained within the pores to contact the substrate in the process stream. The second opening can be larger or smaller or about the same in size than the first opening. In some embodiments, the pores are large enough to contain liposomes therein.

FIG. 3 illustrates, in a schematic, the electrochemical reduction of NAD to NADH2 and the provision of NADH2 to a synthetic mitochondrion which utilizes the NADH2 to transform ADP to ATP via the ETC and ATP synthase. The thus formed ATP is utilized by the cell-free system to make the desired product. The synthetic mitochondrion comprises the ATP synthase, and at least one of the other components of the ETC sufficient to generate a proton gradient across the biological membrane, or membrane of the prepared liposome, of the synthetic mitochondrion, thus providing motive power to the ATP synthase. The synthetic mitochondrion is separated from the process stream which recirculates through the cathode chamber by a membrane permeable to NADH2, but not permeable to the enzymes forming the cell free system, nor the synthetic mitochondrion.

FIG. 3 legends:
100 Electrochemical bioreactor module (EBM) system
101 anode
102 proton permeable membrane (PEM)
103 cathode
200 first recirculation loop containing NADH2 leaving the cathode chamber, and NAD returning to the cathode chamber
800 second recirculation loop transferring ATP produced by the synthetic mitochondrion and providing it to the cell-free system 600 requiring ATP, and transferring ADP produced by the cell free system back to the synthetic mitochondrion of regeneration to ATP
801 a first membrane separating the first recirculation loop 200 from the second recirculation loop 800
802 synthetic mitochondrion containing the ETC components and ATP synthase, present in first recirculation loop 800
803 oxygen provided to capture electrons at the end of the ETC (ETC Complex IV), and form water
803 ADP, inorganic phosphate, and ATP present in the second recirculation loop 800
500 an optional second membrane separating the cell-free system 600 from the second recirculation loop 800
600 the cell-free system which requires ATP
700 process stream containing starting materials input to the cell-free system 600, and the product produced by the cell-free system

FIG. 4 illustrates, in a schematic, the electrochemical reduction of NAD to NADH2 and the provision of NADH2 to a synthetic mitochondrion which is contained within the matrix of a membrane that can be permeated by NADH2, but not by the enzyme forming the cell-free system. The synthetic mitochondrion uses the NADH2 to regenerate ATP from ADP, and the thus formed ATP is utilized by the cell-free system to make the desired product.

FIG. 4 legends:
100 Electrochemical bioreactor module (EBM) system
101 anode
102 proton permeable membrane (PEM)
103 cathode
200 first recirculation loop containing NADH2 leaving the cathode chamber, and NAD returning to the cathode chamber
300 a first membrane separating the first recirculation loop 200 from the second recirculation loop 400
301 synthetic mitochondrion containing the ETC components and ATP synthase, which is held within the matrix of membrane 300
302 oxygen provided to capture electrons at the end of the ETC (ETC Complex IV), and form water
400 second recirculation loop transferring ATP produced by the synthetic mitochondrion and providing it to the cell-free system 600 requiring ATP, and transferring ADP produced by the cell-free system back to the synthetic mitochondrion of regeneration to ATP
500 an optional second membrane separating the cell-free system from the second recirculation loop
600 the cell-free system which requires ATP
700 process stream containing starting materials input to the cell-free system 600, and the product produced by the cell-free system

### EXAMPLES

### Example 1. Preparation of Lipid, Polymer and Hybrid Liposomes

Liposomes can be prepared from soy L-α-phosphatidylcholine (95%, Avanti Polar Lipids) dissolved in 2:1 chloroform-methanol (V/V) and stored at −20° C. until use. First, 10 mg of dissolved lipid can be deposited into a round bottom glass vial and the solvent can be evaporated under a gentle stream of nitrogen. Thin lipid film can be rehydrated with a vesicle buffer, containing 20 mM HEPES (pH 7.5), 2.5 mM MgSO4, 50 mg ml−1 sucrose and resuspended at a final lipid concentration of 10 mg ml−1 by gentle vortexing. Suspension of multilamellar vesicles (MLVs) can be subjected to 7 freeze-thaw cycles (1 min in liquid nitrogen, then water bath, 35° C., until thawed completely, followed by 30 s vortexing). Finally, the size and lamellarity of liposomes in the suspension can be unified by the extrusion (21 times) of lipid suspension through 100 nm pore (polycarbonate membrane, Whatman).

Thin film for hybrid liposomes can be prepared from the lipid/polymer mixture consisting of 30 mol % of soy PC and 70 mol % of polymer. Hybrid liposomes can be then formed according to the protocol described above for liposomes.

Polymer liposomes can be prepared from PDMS-g-PEO dissolved in 2:1 chloroform-methanol (V/V) and stored at room temperature. Thin polymer film can be formed by solvent evaporation under nitrogen and the polymer can be resuspended as mixed polymer/detergent polydispersed liposomes at 10 mg ml−1 polymer concentration in vesicle buffer, supplemented with either sodium cholate, sodium deoxycholate or octy glucoside. Freeze-thaw cycles can be omitted. Monodispersed mixed polymer/detergent liposomes can be formed with extrusion (21 times) through 100 nm pore.

### Example 2. Co-Reconstitution of Proteins into Lipid, Polymer and Hybrid Liposomes

An optimized protocol for the reconstitution of membrane proteins is as follows. Briefly, aiming at a theoretical protein-per-liposome ratio of approx. 1 ATP synthase and 2-10 ETC, 0.14 μM of preformed liposomes (100 μL) can be mixed with 0.14 μM ATP synthase and 0.70 μM of ETC in the presence of 0.4% sodium cholate. The reconstitution mixture can be incubated at room temperature for 30 min with mild agitation followed by the detergent removal using a pre-packed size exclusion column (PD MiniTrap G-25, GE Healthcare). In order to determine the lower production limit of the energy regeneration system, 1 ATP synthase per liposome can be reconstituted. With increasing number of enzymes per vesicle, higher ATP synthesis rates can readily be achieved. To account for the random orientation of the reconstituted ETC, an excess amount of enzyme can be integrated into nanocontainers (5 per liposome).

To accommodate the specificities of the reconstitution protocols developed for hybrid and polymer liposomes, the liposomes can be additionally reconstituted with two other mediating-detergents, sodium deoxycholate and octyl glucoside. A broad concentration range of all detergents (0.2-0.8% with 0.2% increments) can be screened for optimal reconstitution conditions. Furthermore, alternative detergent removal methods, namely detergent removal by Bio-Beads SN-2 (Bio-Rad) and by dialysis can be tested. For the detergent removal by Bio-Beads, after the 30 min incubation period, the reconstitution mixture can be supplemented with 100 mg of beads in a single step and can be incubated at room temperature on a rocking platform for 2 h. After that, beads can be pelleted and the supernatant (proteoliposomes) can be collected. The dialysis can be performed in QuixSep dialysis capsules (Carl Roth). Dialysis membrane (Spectra/Por 7, 8 kDA) can be stretched across the dialysis capsule and the samples can be dialyzed overnight at 4° C. against 100 ml of vesicle buffer supplemented with 100 mM KCl. Detergent removal by Bio-Beads led to highest activity of both reconstituted enzymes and can be therefore used for all subsequent experiments.

Hybrid liposomes can be co-reconstituted following the protocol described above, for the formation of proteoliposomes, with slight modification. Preformed hybrid liposomes can be used for reconstitution mixture instead of preformed liposomes. The detergents can be removed by Bio-Beads, but, to the reconstitution mixture, the beads can be added in 3 subsequent additions, 15 mg of beads each, followed by the 30 min incubation period, at room temperature, on a rocking platform.

The co-reconstitution of polymersomes can be different from the co-reconstitutions of liposomes and hybrids and should be explicitly followed in order to obtain the highest activity of ATP synthase (ETC remains highly active following either of two described protocols). First, 0.14 μM ATP synthase can be added to preformed polymer/detergent mixed liposomes and the reconstitution mixture can be incubated for 15 min at room temperature with occasional gentle agitation. Then, 0.70 μM of ETC can be added and the mixture can be incubated for additional 20 min under same conditions. Detergents can be removed by Bio-Beads in a same way as described above for hybrid liposomes.

### Example 3. Determination of Respiration-Driven ATP Production

Measurements of respiration-driven ATP production can be performed as follows. To a solution containing 480 μl of measurement buffer (20 mM Tris-PO4 (pH 7.5), 10 μl of luciferin/luciferase assay (CLSII, prepared according to manufactures protocol), 2 μl of ADP (8.45 mM stock, ultra-pure) and 1 μl DTT (1M stock)), 10 μL of liposomes can be added and a baseline can be recorded. The reaction can be started by the addition of 1 μl of ubiquinone Q1 (10 mM stock) and synthesis of ATP can be recorded. At the end of each measurement, 3 μl of ATP (5 μM stock) can be added to normalize the signal against a defined ATP amount. The ATP production rates can be reported as the average of at least 3 separate preparations (each measured in 3 replicates), with standard deviation.

### Example 4. Various ATP Regeneration Systems

### Example 4a

As a non-limiting example, the electrochemical bioreactor includes a first solution of NAD recirculating through the cathode chamber. In the recirculation loop, a first membrane is provided that has pores sufficient to allow NAD and NADH2 to permeate the first membrane. Optionally, FAD may also be supplied for electrochemical reduction to FADH2. On the other side of the first membrane, a second recirculating process stream is present.

A synthetic mitochondrion can be prepared according to methods known in the art, such as the liposomes based on the graft copolymer poly(dimethylsiloxane)-graft-poly(ethylene oxide) (PDMS-g-PEO) as disclosed in Otrin et al. (Nano Lett. 2017, 17, 6816-6821), incorporated herein by reference in its entirety, and added to the second recirculating process stream. This synthetic mitochondrion contains the four ETC components illustrated in FIG. 2, plus the ATP synthase. A cell-free system for the production of a peptide from individual amino acids is prepared, and also added to the second recirculating process stream. The individual amino acids are provided to the second recirculating process stream, with ADP. The electrochemical bioreactor is energized, and NAD in the first recirculating stream is reduced to NADH2. The NADH2 permeated the first membrane, and interacts with the synthetic mitochondrion in the second process stream, providing reducing equivalents to the ETC of the synthetic mitochondrion. The NADH2 is thus oxidized to NAD, permeates the first membrane in the opposite direction and returns to the first recirculating process stream, where it enters the cathode chamber as is reduced to NADH2. The synthetic mitochondrion in the second recirculating process stream, having been charged by the NADH2 and having generated a proton gradient, transforms ADP to ATP. The thus produced ATP is distributed in the second process stream, where it provided the necessary chemical potential energy to the peptide synthesis components of the cell free system, being transformed back to ADP in the process. The peptide synthesis components of the cell-free system condense the individual amino acids present in the second process stream to form the desired peptide product. The ADP is transformed back to ATP by the synthetic mitochondrion. Oxygen is provided proximal to the synthetic mitochondrion to accept electrons from the end of the ETC at complex IV.

The system is prepared as in Example 1, but without the ETC complex II present in the synthetic mitochondrion, and no FAD/FADH2 is provided to the system. Other reduced chemical species such as NAD(P)H2 and FMNH2 can be used.

### Example 4b

The system is prepared as in Example 1, but without either of ETC complex I nor the ETC complex II. The electrochemically generated NADH2 is used to reduce ubiquinone or other suitable quinone to the reduced quinol species, which transfers electrons to ETC complex III.

### Example 4c

The system is prepared as in Example 1, but the synthetic mitochondrion contains only ETC complex IV and the ATP Synthase. An electron carrier or mixture of electron carriers comprising at least one of NADH2, a quinol, and a thiol capable of oxidation, for example dithiothreitol (DTT) in any proportion can be used to provide electrons. Electrons are transferred to the ETC complex IV via at least one of the electron carriers.

It will be clear to those skilled in the art of bioreactor design that in each of the examples given, a series of recirculation loops can be provided, each contacting the adjacent recirculating loops via a suitably permeable membrane.

It will be clear to those skilled in the art of electrochemistry that different electron carriers can be provided, as long as the redox potential of the reduced from of the provided electron carrier is sufficient to provide electrons to ETC complex IV, either directly or via other components of the ETC. It will further be clear that such flexibility of provided electron carriers allows different voltages to be used in the electrochemical cell in the system.

It will be clear to those skilled in the art of synthetic biology, and particularly cell-free systems, that providing reducing equivalents for driving the regeneration of ATP may be used to drive other reactions that require ATP, for example, phosphorylation of sugars or lipids for producing surfactants or other commercially valuable materials.

Those skilled in the art will also recognize that other nucleoside triphosphates can be produced by using the enzyme nucleoside-diphosphate kinase (NDK) to generate a different nucleoside triphosphate from ATP, leaving ADP as the by-product. For example, the nucleoside triphosphate GTP can be produced by the action of NDK, consuming ATP in the process thusly:

ATP+GDP [symbol] ADP+GTP,

with the ADP being recycled back to ATP by the present disclosure. This permits the same ATP regeneration system to be used to produce GTP for use in sugar phosphorylation or other reactions where phosphorylation by a nucleoside triphosphate is required, but ATP is not used by the specific phosphorylation enzymes.

### EQUIVALENTS

The present disclosure provides among other things novel methods and devices for providing reducing equivalents to biological systems. While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

### INCORPORATION BY REFERENCE

All publications, patents and patent applications cited above are incorporated by reference herein in their entirety for all purposes to the same extent as if each individual publication or patent application were specifically indicated to be so incorporated by reference.

What is claimed is:

1. A cell-free composition for regenerating adenosine triphosphate (ATP) comprising:
a reduced chemical species for delivering electrons to an electron acceptor, wherein said reduced chemical species is electrochemically generated by an electrochemical device;
an electron transport chain (ETC) comprising the electron acceptor and ETC complex IV, wherein the electron acceptor is selected from the group consisting of ETC complex I, ETC complex II, ETC complex III, ubiquinone and cytochrome c, wherein said ETC is capable of generating a proton gradient across a lipid bilayer;
an ATP synthase capable of utilizing the proton gradient across the lipid bilayer to produce ATP from adenosine diphosphate (ADP); and
a liposome enclosed by the lipid bilayer to retain protons within the liposome, wherein the ETC and the ATP synthase are associated with the lipid bilayer.

2. The cell-free composition of claim 1 wherein the reduced chemical species is one or more of NADH2, NADPH2, FMNH2, FADH2 and/or ubiquinol.

3. The cell-free composition of claim 1 wherein the reduced chemical species is one or more of NADH2, NADPH2 and/or FMNH2, and wherein the electron acceptor is ETC complex I.

4. The cell-free composition of claim 1 wherein the reduced chemical species is FADH2, and wherein the electron acceptor is ETC complex II.

5. The cell-free composition of claim 1 wherein the reduced chemical species is ubiquinol, and wherein the electron acceptor is ETC complex III.

6. The cell-free composition of claim 1 wherein the ETC has been reconstituted into the liposome to at least partially embed in the lipid bilayer, with the electron acceptor accessible to the reduced chemical species that are present outside the liposome.

7. The cell-free composition of claim 1 wherein the ATP synthase has been reconstituted into the liposome to at least partially embed in the lipid bilayer, and wherein the ATP synthase is accessible to the ADP that is present outside the liposome.

8. The cell-free composition of claim 1 wherein the lipid bilayer is impermeable to protons.

9. The cell-free composition of claim 1 wherein the liposome is artificially prepared.

10. The cell-free composition of claim 1 wherein the ETC and/or ATP synthase are recombinantly produced.

11. A device for regenerating adenosine triphosphate (ATP), comprising:
an anode contained in an anode chamber and a cathode contained in a cathode chamber;
deionized water in the anode chamber in contact with the anode;
a proton permeable membrane that separates the anode and cathode chambers;
a liquid phase in the cathode chamber continuously in contact with the cathode, said liquid phase comprising an electron transfer mediator (ETM) capable of undergoing cyclic reduction and oxidation, wherein, once reduced at the cathode, the ETM is reduced into reduced chemical species that can transfer electrons to an electron acceptor of an electron transport chain (ETC); wherein the electron acceptor is selected from the group consisting of ETC complex I, ETC complex II, ETC complex III, ubiquinone and cytochrome c; wherein the ETC comprising the electron acceptor and ETC complex IV;
a synthetic mitochondrion comprising an enclosed lipid bilayer, the ETC, and an ATP synthase; wherein said ETC is capable of generating a proton gradient across the lipid bilayer; wherein said ATP synthase capable of utilizing the proton gradient across the lipid bilayer to produce ATP from adenosine diphosphate (ADP); wherein the enclosed lipid bilayer is capable of retaining protons therein;
a process stream containing a substrate capable of using the generated ATP for producing a desired product; and
an external power source providing a voltage between the anode and the cathode.

12. The device of claim 11 wherein the ETC and the ATP synthase are reconstituted to be associated with, preferably at least partially embedded within, the lipid bilayer.

13. The device of claim 11 further comprising a membrane located between the cathode and the process stream, wherein said membrane supports the synthetic mitochondrion and permits interfacial contact between the liquid phase and the process stream, while preventing substantial mixing of the liquid phase and the process stream and preventing the process stream from substantially contacting the cathode.

14. The device of claim 13, wherein the membrane is configured to partially associate with the synthetic mitochondrion.

15. The device of claim 13, wherein the membrane is configured such that the synthetic mitochondrion is partially or completely embedded within the membrane.
